(19) United States
(12) Patent Application Publication
(10) Pub. No.: US 2007/0156016 A1
(43) Pub. Date: Jul. 5, 2007

(54) Method and system for communication with an ingestible imaging device

(76) Inventors: Ido Betesh, Haifa (IL); Micha Nisani, Nesher (IL); Pesach Pascal, Nesher (IL)

Correspondence Address: PEARL COHEN ZEDEK LATZER, LLP, 1500 BROADWAY 12TH FLOOR, NEW YORK, NY, 10036, US

(21) Appl. No.: 11/319,803

(22) Filed: Dec. 29, 2005

(51) Int. Cl. A61B 1/00

(52) U.S. Cl. 600/102; 370/315

(57) ABSTRACT

A system and method for detecting and recording interference in an RF communication channel for an ingestible imaging device is provided. During a period when transmission in a communication channel may be idle, interference levels may be detected and recorded. Changes in transmission levels may be altered based on the detected interference level. Transmission may be delayed due to detected levels of interfering signals.

### FIELD OF THE INVENTION

The present invention relates to ingestible imaging devices, and more particularly to communication between an ingestible imaging device and an external recorder.

### BACKGROUND OF THE INVENTION

In-vivo imaging devices for diagnosis of the gastrointestinal (GI) tract such as for example ingestible imaging capsules may wirelessly transmit and receive signals from an external recording device. A diagnosis procedure utilizing a known ingestible imaging capsule may involve a patient wearing a portable recording device with one or more antennas to receive and record data transmitted from the ingested capsule for a period, for example a period of eight hours, in which the capsule may be passing through the GI tract. Images of the GI tract may be captured by an ingested capsule and transmitted, for example by RF signals to the recording device. A patient may be able to carry on with daily activities, e.g. going to work, shopping, etc while undergoing such a procedure. Other RF transmitting devices, for example, other ingestible imaging devices, cell phones or remote control for automobiles, etc may at times interfere with proper reception and/or transmission between the capsule and the recorder. Data that may be important in diagnosis may be lost or corrupted due to interference.

### SUMMARY OF THE INVENTION

According to embodiments of the present invention, there is provided a system and method for detecting and recording interfering signals and/or data pertaining to interference levels that may interfere with proper reception and transmission between an ingestible imaging device and a recorder. According to one embodiment of the present invention, interference may be detected in a first frequency range, for example the frequency range of the wireless communication channel and/or line for transmitting signals from the ingestible device to the external recorder. According to another embodiment of the present invention, interference may be detected in a second frequency range, for example the frequency range of the wireless communication channel for transmitting signals from the external recorder to the ingestible imaging device. Detection of interfering signals may be performed by an interference sensor that may be contained in the ingestible imaging device and/or in the external recorder. An indicator may be operated upon detection of interfering signal whose strength may be above a defined threshold.

### BRIEF DESCRIPTION OF THE DRAWINGS

The present invention will be understood and appreciated more fully from the following detailed description taken in conjunction with the drawings in which:

FIG. 1 is a simplified conceptual illustration of an in-vivo imaging system with an interference sensor for detecting potential inferring signals according to a first embodiment of the present invention;

FIGS. 2A and 2B are timing diagram of transmission and reception of signals according to embodiments of the present invention;

FIG. 3 is a simplified conceptual illustration of an in-vivo imaging system with an interference sensor for detecting potential inferring signals according to a second embodiment of the present invention;

FIG. 4 is a simplified conceptual illustration of an in-vivo imaging system with an interference sensor for detecting potential inferring signals according to a third embodiment of the present invention;

FIG. 5 is a simplified conceptual illustration of an in-vivo imaging system with an interference sensor for detecting potential inferring signals according to a fourth embodiment of the present invention;

FIG. 6 is a timing diagram of transmission and reception of signals according to other embodiments of the present invention;

FIG. 7 is a flow chart describing a method of detecting interference levels in the uplink channel according to embodiments of the present invention; and

FIG. 8 is a flow chart describing a method of detection interference levels in the downlink channel according to embodiments of the present invention.

It will be appreciated that for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well-known features may be omitted or simplified in order not to obscure the present invention.

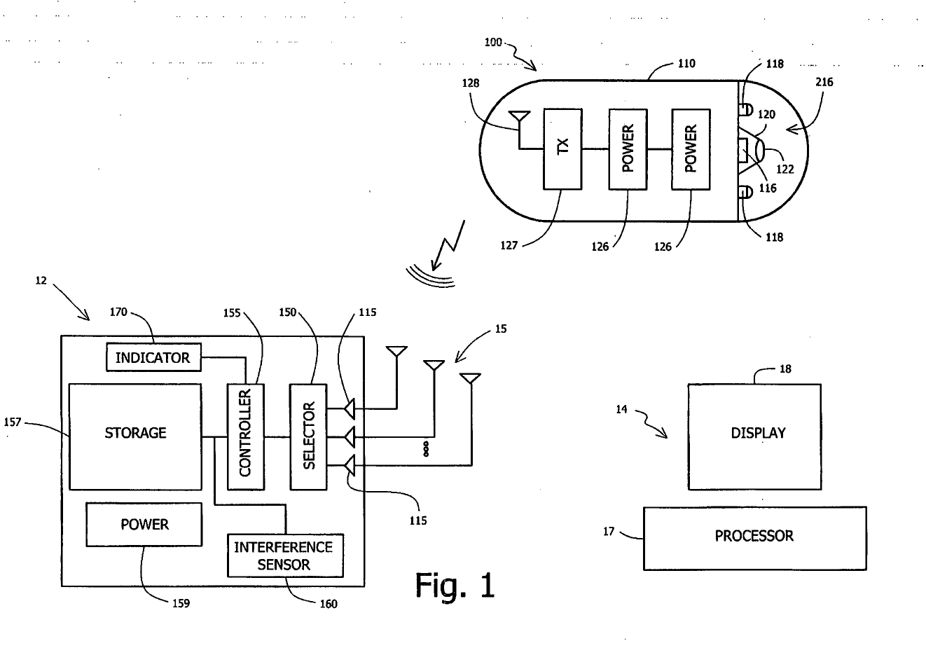

Reference is made to FIG. 1, showing a simplified conceptual illustration of an in-vivo imaging system with an interference sensor for detecting potential interfering signals and the strength of the interfering signals according to an embodiment of the present invention. Ingestible device 100 may be an autonomous in-vivo sensor, for example, an in-vivo imaging device for gathering data in-vivo. Data, for example image data and other data may be transmitted by wireless connection, e.g. wireless communication channel, from the ingestible device 100 to an external recorder 12. Recorder 12 may function as a RF receiver/recording unit with one or more receiving antennas 15. Data captured by ingestible device 100 and received by recorder 12 via antennas 15 and may be, for example downloaded to workstation 14 for processing, analysis, and display, for example, with processor 17 and display unit 18. Downloading and/or processing in workstation 14 may occur off-line for example after the recorder completed recording of data from the ingestible device 100, or may occur in real-time and/or in pseudo real time. In one embodiment of the present invention, recorder 12 and workstation 14 may be integrated into a single unit, for example, may be integrated into a single portable unit. In other embodiments recorder 12 may be include separate recording unit and a separate receiving unit. In yet another embodiment of the present invention, recorder 12 may include display capability, for example recorder 12 may include an on-line viewer for viewing information and/or images, for example information and/or images transmitted by ingestible device 100. In another embodiment, processing and/or analysis may be performed at least partially within the recorder 12.

Ingestible device 100 may include a sensing device such as for example an imaging unit 216 within an outer covering or housing 110, constructed and operative in accordance with an embodiment of the invention. Housing 110 may be, for example, spherical, ovoid, or any other suitable shape and may be at least partially transparent. Imaging unit 216 may typically include at least one imager 116, which may be or may include a charge coupled device (CCD), a complementary metal oxide semiconductor (CMOS) imager, another suitable solid-state imager or other imagers. In addition imaging unit 216 may include, for example a lens 122 and a lens holder 120 as well as one or more (e.g., a pair, a ring, etc.) illumination sources 118, such as for example, light emitting diodes (LEDs), which may illuminate the areas to be imaged by the imager 116. Other positions for imager 116, illumination sources 118 and other components may be used and other shapes of a housing 110 may be used.

In embodiments of the present invention ingestible device 100 may include and/or contain one or more power units 126, a transmitter 127, e.g. an RF transmitter, and one or more antennas 128 for transmitting data. Power unit 126 may include one or more batteries and/or other suitable power sources. In another example power unit 126 may include a power induction unit that may receive power from an external source. In one example, transmitter 127 may include control capability, for example transmitter 127 may be or include a controller for controlling various operations of ingestible device 100, although control capability or one or more aspects of control may be included in a separate component such as for example circuit board or other circuitry included in ingestible device 100. Transmitter 127 may typically be included on an Application Specific Integrated Circuit (ASIC), but may be of other constructions. Ingestible device 100 may include a processing unit separate from transmitter 127 that may, for example, contain or process instructions.

Ingestible device 100 may be inserted in-vivo, for example by swallowing and may move through the GI tract by means of natural peristaltic motion to be finally naturally excreted through the rectum. In other examples, ingestible device 100 may be, for example, fixed and or positioned within a body lumen and may for example be removed at the termination of a diagnostic procedure. Ingestible device 100 and recorder 12 may include components and operate similarly to the imaging systems described, for example, in U.S. Pat. No. 5,604,531 to Iddan, et al. and/or in US Patent Application Publication Number 20010035902, entitled “Device and system for in vivo imaging”, published on Nov. 1, 2001 both of which are assigned to the common assignee of the present application and both of which are hereby fully incorporated by reference.

Ingestible device 100 may be a capsule or other unit that does not require wires or cables external to ingestible device 100, for example, to receive power or transmit information. For example, power may be provided by an internal battery. Other embodiments may have other configurations and capabilities. For example, components may be distributed over multiple sites or units. Control information may be received by wireless communication from an external source. Transmission of data, for example image data may be periodically transmitted to a recorder 12. For example, image data captured, for example image data of a single image frame, may be transmitted every 500 msec to recorder 12 at a frequency in the order of magnitude of 400 MHz, e.g. 400-450 MHz. Other capturing and transmitting rates of image frame data may be used, for example in the range of 1 to 40 frames per second.

External recorder 12 may receive transmissions from ingestible device 100, such as, for example, a stream of wireless communication frames, from ingestible device 100 through one or more antennas 15. According to one embodiment of the present invention, one or more antennas 15 may be positioned around or in proximity to a patient swallowing ingestible device 100. For example, one or more antennas 15 may be placed on the abdominal region of the patient. Alternatively, antennas 15 may be mounted inside recorder 12. Other configurations of antennas 15 may be used. Signals and/or data, for example image data and control data transmitted by the ingestible device 100, as well as other signals from the surrounding environment may be picked up by the one or more antennas 15. Recorder 12 may incorporate storing capability to store for example, data received via antennas 15. Recorder 12 may communicate with workstation 14, for example, via hard-wired medium 14. Data and/or signals received by recorder 12 may be processed and displayed in workstation 14.

For simplicity of explanation without the loss of generality, it is assumed that ingestible device 100 may contain one transmitting antenna, e.g., transmitting antenna 128, and antenna 128 may transmit a signal that may be received by a plurality of receiving antennas 15 resulting in a plurality of received signals. Interference from the surrounding environment may also be picked up by receiving antennas 15 and may at times distort the data signal transmitted by antenna 128 from ingestible device 100 and picked up by antennas 15.

According to one embodiment of the present invention, recorder 12 may include a signal selector 150 that may select or may produce an output signal that may be constructed from the plurality of received signal received from one or more antennas 15, a control unit 155 to control operation of recorder 12 and demodulate the received signals, an interference sensor 160 to detect a strength of a signal, a storage unit 157 to store data received, and a power unit 159 to power recorder 12. One or more amplifiers 115 may amplify signals received by antennas 15. Signal selector 150, control unit 155, amplifiers 115 and interference sensor may be incorporated in a single unit or may be distributed in more than one unit. Signal selector 150 may be for example a multiplexer or other suitable signal selection device. The selection of the received signals may be based upon some pre-defined criteria such as, for example, relative signal strength or other criteria. In one example, the received signal with the highest signal strength may be selected. In another example, a two or more signals, for example two or more relatively strong signals may be selected and/or combined, for example based on their relative signal strengths. Other methods of selection may be implemented. Control unit 155 may include analog to digital conversion capability, processing capability and control capability to control the operation of recorder 12 and storage of data received. Storage unit 157 may store data received by recorder 12 and may be for example a flash memory card or other suitable memory unit Interference sensor 160 may include for example a signal strength detector, e.g. a Received Signal Strength Indicator (RSSI) to detect the strength of a signal received by antennas 15 and/or selected by signal selector 150 and may be contained, for example, within the housing of the recorder 12. Detected signal strength and or data pertaining to the interfering signal, e.g. interference data may be stored in storage unit 157. In another example indication that a signal may be above one or more defined thresholds, e.g. a Boolean parameter, may be saved in storage unit 157. In another example, any function of the RSSI may be saved to storage unit 157. Indicator 170 may alert a user, e.g. the patient or the health profession that the patient is situated in an area where interference signals are being picked up the recorder 12. For example, indicator 170 may be a visual indicator, e.g. an LED, an audio indicator, e.g. a buzzer or other indicator e.g. a vibrator that may give indication to the patient that he is situated in an area where interference signals are being picked up the recorder 12. In reaction to the indication provided by indicator 170, the patient may move away from or possibly cease operation off an interfering RF transmitter.

In one embodiment of the present invention control unit 155 may prompt the recorder 12 to receive signals that may be present in the surrounding environment during a period when no transmission from the capsule is expected. Signal selector 150 may for is example amplify the signals picked up with one or more amplifiers 115 and select one or more signals picked up from the plurality of antennas 15. In one example, the received signal with the highest signal strength may be selected. In another example, a two or more signals, for example two or more relatively strong signals may be selected and/or combined, for example based on their relative signal strengths. Other methods of selection may be implemented. Interference sensor 160 may determine the signal strength. The signal strength detected by interference sensor 160 may be recorded in storage unit 157. Signal strengths above a defined threshold, e.g. determined by a threshold detector, may prompt indicator 170 to give indication to the patient that the receiver is picking up high levels of interference. Alternatively and/or in addition, a classification of the interference picked up may be recorder. In one example, the classification may be stored in storage unit 157 to indicate levels of interference. For example a first level and/or degree of interference, detected for example by a threshold detector, may indicate that an interfering signal was above a first defined threshold, a second level may be stored in storage unit 157 to indicate that an interfering signal was above a second defined threshold. More or less than 2 threshold markers may be used. The first threshold may indicate that extra processing may be required to decipher the signal, e.g. based on an estimated signal to noise ration (SNR). The second level may indicate that the interfering signal is at a level that makes the transmitted signal unusable. Levels of the interference signal may be used to process the signals transmitted by ingestible device 100. For example, different levels and forms of processing may be used on the signals transmitted by ingestible device 100 and received by recorder 12 based on the interference levels detected in a time proximal to the time that the transmitted signal from ingestible device 100 was picked up by recorder 12. In other examples, signal interference levels may be detected a plurality of times between transmissions from the ingestible device 100 to the recorder 12 and interference level data accumulated over time may be used to estimate the inference level during the time of transmission from the ingestible device 100 to the recorder 12.

According to one example, controller 155 may alter and/or adjust one or more parameters for demodulation and storing of data received by ingestible device 100 based on the level of interference detected. For examples, data received may not be stored or may only be partially stored in storing unit 157 if received during a period estimated to have a high level of interference. Other parameters may be adjusted.

Data stored in recorder 12 may be downloaded to workstation 14 for further processing and presentation. Interference levels measured may be used during the processing of data stored in storage unit 157. For example, if high interference levels were picked up close to period where a particular image frame or a plurality of image frames were transmitted, those particular frames may be removed from the image stream. In other examples, the level of interference may indicate the level of processing required and the SNR. For low levels of interference, less processing may be required, or a specific processing may be required. For medium levels of interference more processing may be required, or a particular processing may be required.

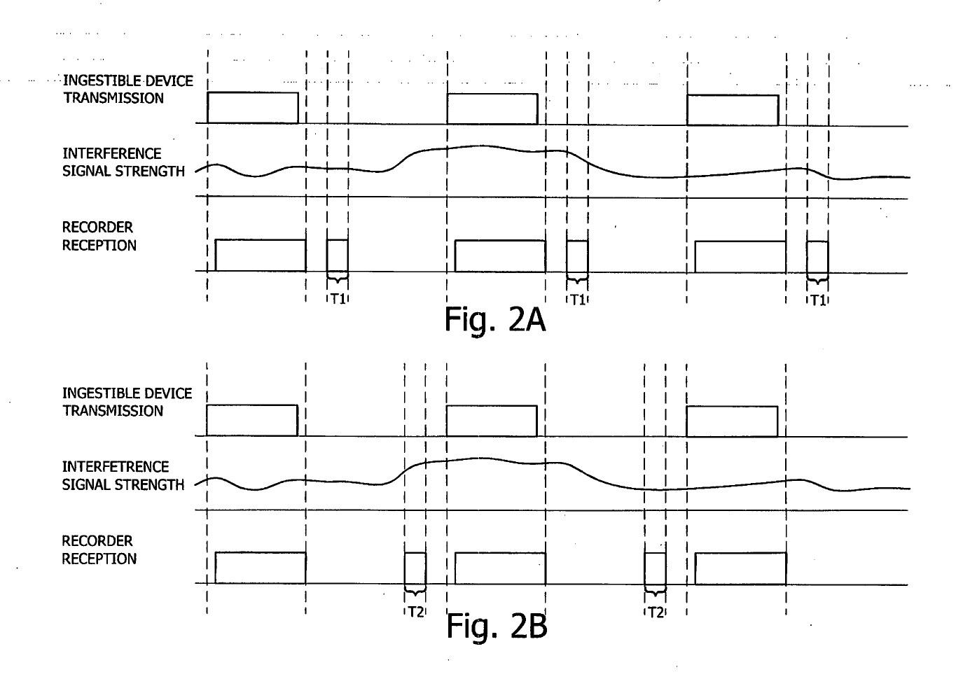

Reference is now made to FIGS. 2A and 2B showing time diagrams of data transmission from ingestible device 100 and signal reception by the recorder 12. According to one embodiment of the present invention, the ingestible device 100 may transmit data at a defined range of frequencies to the recorder 12, for example every 500 msec at a frequency range in the order of magnitude of 400 MHz. Interference signals in that frequency range, e.g. with an example signal strength over time indicated in FIGS. 2A and 2B may be present in the vicinity of recorder 12. The recorder 12 may receive and record the data transmitted by ingestible device 100 together with interference signals. After a defined period that may be prompted by the end of reception, the recorder 12 may receive and store interference signal in the defined frequency range during a period T1 while no data is transmitted by ingestible device 100. In an alternate embodiment, shown in FIG. 2B reception of interference signals may occur in proximity and prior to anticipated reception, for example during a period T2. In other embodiments, interference signals may be monitored in other periods or multiple periods between receptions of signals from ingestible device 100, or may be monitored continuously between receptions. Interference signals may be stored together with the data received from ingestible device 100 and/or may be processed before storing.

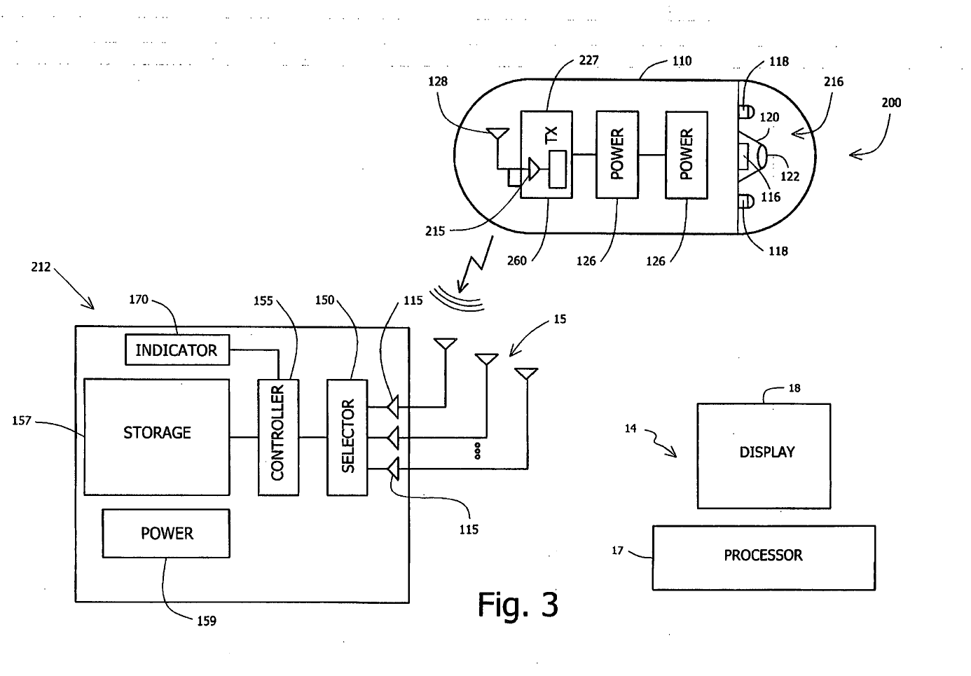

Reference is now made to FIG. 3 showing a simplified conceptual illustration of an in-vivo imaging system with an interference sensor for detecting potential inferring signals according to a second embodiment of the present invention. In the system according to the second embodiment, elements having the same reference numerals and names as those of the first embodiment have the same configurations and actions as those of the first embodiment unless otherwise specified in the following.

Ingestible device 200 may be an autonomous in-vivo sensor that may transmit data to a recorder 212, for example a recorder including an RF receiver having one or more receiving antennas 15. Data, for example image data and other data may be transmitted by wireless connection from the ingestible device 200 to an external recorder 212. Recorder 212 may function as a RF receiver/recording unit with one or more receiving antennas 15. Data captured by ingestible device 200 and received by recorder 212 via antennas 15 may be, for example downloaded to workstation 14 for processing, analysis, and display, for example, with processor 17 and display unit 18.

Ingestible device 200 may include a sensing device such as for example an imaging unit 216 within an outer covering or housing 110. Imaging unit 216 may typically include at least one imager 116. In addition imaging unit 216 may include, for example a lens 122 and a lens holder 120 as well as one or more (e.g., a pair, a ring, etc.) illumination sources 118. Ingestible device 200 may include and/or contain one or more power units 126, a transmitter 127, e.g. an RF transmitter, and one or more antennas 128 for transmitting and/or receiving data. Transmitter 127 may include control and or processing capability, for example transmitter 127 may be or include a controller for controlling various operations of ingestible device 200, although control capability or one or more aspects of control may be included in a separate component such as for example circuit board or other circuitry included in device 200.

External recorder 212 may be able to receive transmissions, such as, for example, a stream of wireless communication frames, from ingestible device 200 through one or more antennas 15. Recorder 212 may include a signal selector 150 that may select or may produce an output signal that may be selected from the plurality of received signals, a control unit 155 to for example control operation of recorder 212 and demodulate the received signals, a storage unit 157 to store data received, and a power unit 159 to power recorder 212.

According to the second embodiment of the present invention, an interference sensor 260 may be included within ingestible device 200. For example, an antenna 128 may, e.g., during an idle time or period of transmission, receive and/or pick up signals in the frequency range of antenna 128 from the surrounding environment that may be interfering signal. The strength of signals picked up, e.g. during the idle period of transmission, may be detected by an interference sensor 260. Interference sensor 260 may be for example a RSSI component to detect the strength of a signal received by antenna 128. An amplifier 215 may be used to amplify the received signal prior to detecting the signal strength. In one example the amplifier 215 and/or the interference sensor 260 may be integral to the transmitter 127. In alternate examples, the interference sensor 260 may be separate or integral to other components, e.g. the imager 116 or other components in the ingestible device 200. Data detected by interference sensor 260 may be transmitted to receiver 212 during the regular transmission period of ingestible device 200. For example, signal strength data may be sent together with an image frame transmitted from ingestible device 200. For example, signal strength data may be included as a header or suffix of the transmitted image data. Other methods of incorporating signal strength data with data otherwise transmitted from ingestible device 200 may be used. In an alternate example, signal strength data may be transmitted separately. According one example, the signal strength measurement may be transmitted to recorder 212 and the data may be stored in storage unit 157. According to one example, specified threshold levels of interference may be detected by circuitry in recorder 212 and the threshold level may be stored. In another example, determination of threshold levels may be performed in the ingestible device 200 prior to transmission. For example, an indication of interfering signals may only be transmitted for signal interference above a defined threshold. Other suitable methods of detecting, storing and communication the level of interference may be implemented.

Indicator 170 may alert a patient or health profession that the patient is situated in an area where interference signals are being picked up the recorder 212. For example, indicator 170 may be a visual indicator, e.g. an LED, an audio indicator, e.g. a buzzer or other indicator e.g. a vibrator that may give indication to the patient that he is situated in an area where interference signals are being picked up the recorder 212. In reaction to the indication provided by indicator 170, the patient may move away from or possibly cease operation off an interfering RF transmitter.

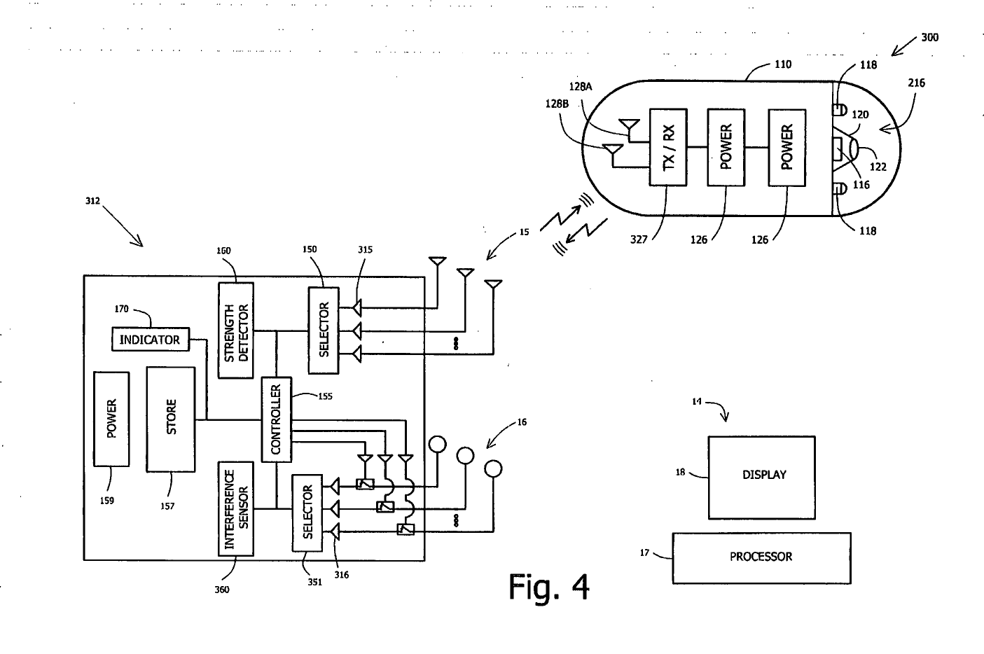

Reference is now made to FIG. 4 showing simplified conceptual illustration of an in-vivo imaging system with an interference sensor for detecting potential inferring signals according to a third embodiment of the present invention. According to the third embodiment of the present invention, ingestible device 300 may transmit and receive data to and from a recorder 312. The communication channel including transmission from the ingestible device 300 to the recorder 312 may be referred to herein as the uplink channel, while the communication channel and/or line including transmission from the recorder 312 to the ingestible device 300 may be referred to herein as the downlink channel. According to one embodiment of the present invention, transmission from ingestible device 300, e.g. the uplink channel and reception to ingestible device 300, e.g. the downlink channel may be performed at different frequencies and therefore separate antennas may be used for the uplink channel and the downlink channel. For example, a first frequency range may be used for the communication line of the uplink channel, and a second frequency range may be used for the communication line of the downlink channel. According to one example, the uplink channel may be in the order of magnitude of approximately 400 MHz while the downlink channel may be in the order of magnitude of approximately 10 MHz, e.g. 10-15 MHz. Interference signals may be detected for each of the channels and/or communication frequencies, e.g. transmission and reception frequencies. In the system according to the third embodiment, elements having the same reference numerals and names as those of the first embodiment have the same configurations and actions as those of the first embodiment unless otherwise specified in the following.

Ingestible device 300 may be an autonomous in-vivo sensor that may transmit and receive data to and from a recorder 312, for example a recorder including an RF receiver having one or more receiving antennas 15, and an RF transmitter having one or more transmitting antennas 16. In other embodiments of the present invention, a single set of antennas may be used for both uplink and downlink channels. Data captured by ingestible device 300 and received by recorder 312, i.e. the uplink channel may be, for example downloaded to workstation 14 for processing, analysis, and display, for example, with processor 17 and display unit 18. Data transmitted by recorder 312 and transmitted to ingestible device 300, i.e. the downlink channel may include control data that may control the operational and functional state of ingestible device 300.

Ingestible device 300 may include a sensing device such as for example an imaging unit 216 within an outer covering or housing 110. Imaging unit 216 may typically include at least one imager 116. In addition imaging unit 216 may include, for example a lens 122 and a lens holder 120 as well as one or more (e.g., a pair, a ring, etc.) illumination sources 118. Ingestible device 300 may include and/or contain one or more power units 126, a transceiver 327, e.g. an RF transceiver, and one or more antennas 128a for transmitting data and one or more antennas 128b for receiving data, e.g. receiving control data. In other embodiments one or more antennas 128a and/or 128b may be used for transmission as well as reception. In other embodiments, transceiver 327 and or its functionality may be spread among a plurality of components. Transceiver 327 may include control and or processing capability, for example transceiver 327 may be or include a controller for controlling various operations of ingestible device 300, although control capability or one or more aspects of control may be included in a separate component such as for example circuit board or other circuitry included in ingestible device 300.

External recorder 312 may receive transmissions, such as, for example, a stream of wireless communication frames, from ingestible device 300 through one or more receiving antennas 15. Recorder 312 may include a signal selector 150 that may select or may produce an output signal that may be selected from the plurality of received signals, a control unit 155 to control operation of recorder 312 and demodulate the received signals, a storage unit 157 to store data received, and a power unit 159 to power recorder 312.

According to an embodiment of the present invention, recorder 312 may include one or more transmitting antennas 16 to, for example transmit signals to ingestible device 300. Transmitted signals may contain control data or other data that may be for exampled stored in storage unit 157 and/or processed in processing unit 155. During and idle time of reception by the ingestible device 300 and/or a period when the downlink channel may normally be idle, antennas 16 may receive signals, for example signals in the same approximate frequency range of the downlink channel, to determine the level of interference signals that may be present in the surrounding environment in that frequency range. Signal amplifiers 316 may amplify, for example each of the signals picked up each of the antenna 16s, signal selector 351 may select one or more signal or combine one or more signals picked up and interference sensor 360 may determine the signal strength of the picked up and or selected one or more signals. Processor 155 may determine the level of interference based on the output of the interference sensor 360 and may store that information in storage unit 157. According to one embodiment of the present invention, control data transmitted by antennas 16 may include information regarding the signal strength detected and/or the level of interference. Control data may influence the operation of ingestible device 300 by for example increasing the amplification of transmission in the uplink channel, altering the modulation of the signal, and/or delaying transmission of the data packet and or image frame. In one example, if high signal strength is detected, downlink transmission may be avoided. For example, some distributed collision avoidance algorithms such as ALOHA and Carrier-Sense Multiple Access (CSMA) can be used for this purpose, to for example retransmit signals when interference signals may have been detected. In another example, the signal strength may be detected during a period and/or slot of downlink transmission, for example, a slot of downlink transmission in which the recorder may not be transmitting. As such it may be possible to detect if another recorder, besides recorder 312 may be trying to transmit signals to the ingestible device 300. Other changes may be initiated. In other embodiments, the interference or signal strength data may be stored in storage unit 157 for later processing of the image data and may affect the image processing or image frame selection of data presented. In another example indication that a signal may be above one or more defined thresholds, e.g. a Boolean parameter, may be saved in storage unit 157. In another example any function of the signal strength can be saved in storage unit 157.

Interference detection for the uplink channel via antennas 15 may be similar to the discussion presented in reference to FIG. 1 describing the first embodiment of the present invention. In one embodiment of the present invention, control unit 155 may prompt recorder 312 during a period when the uplink channel is normally idle or during an idle time between frames, to pick up signals that may be present in the surrounding environment. Signal selector 150, may for example select the strongest signal picked up from the plurality of antennas 15. Interference sensor 160 may determine the signal strength. The signal strength detected may be recorded in storage unit 157.

Indicator 170 may alert a patient or health profession that the patient is situated in an area where interference signals are being picked up the recorder 312. For example, indicator 170 may be a visual indicator, e.g. an LED, an audio indicator, e.g. a buzzer or other indicator e.g. a vibrator that may give indication to the patient that he is situated in an area where interference signals are being picked up the recorder 312. In reaction to the indication provided by indicator 170, the patient may move away from or possibly cease operation off an interfering RF transmitter.

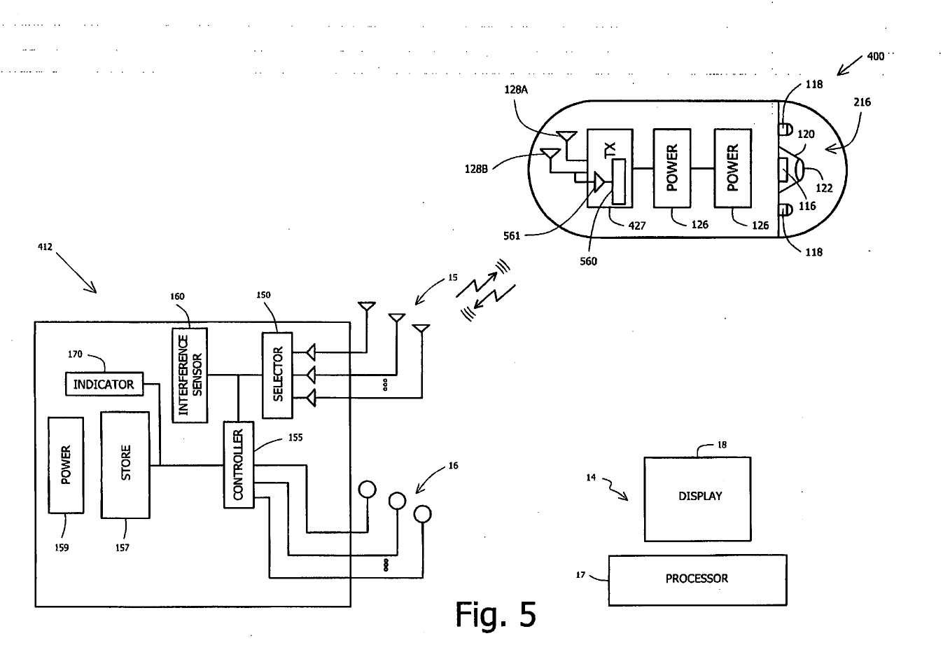

Reference is now made to FIG. 5 showing a simplified conceptual illustration of an in-vivo imaging system with an interference sensor for detecting potential inferring signals according to a fourth embodiment of the present invention. According to the fourth embodiment of the present invention, recorder 412 as well as ingestible device 400 may include transmitting as well as receiving capabilities. According to one embodiment of the present invention, reception and transmission may be performed at different frequencies and therefore separate antennas may be used for uplink and downlink channel as may be described herein. According to one example, the uplink channel may be in the order of magnitude of approximately 400 MHz while the downlink channel may be in the order of magnitude of approximately 10 MHz.

Ingestible device 400 may be an autonomous in-vivo sensor that may transmit data to a recorder 412, for example a recorder including an RF receiver having one or more receiving antennas 15 and an RF transmitter having one or more transmitting antennas 16. Data, for example image data and other data may be transmitted through the uplink channel by wireless connection. Data captured by ingestible device 400 and received by recorder 412 via antennas 15 may be, for example downloaded to workstation 14 for processing, analysis, and display, for example, with processor 17 and display unit 18.

Ingestible device 400 may include a sensing device such as for example an imaging unit 216 within an outer covering or housing 110. Imaging unit 216 may typically include at least one imager 116. In addition imaging unit 216 may include, for example a lens 122 and a lens holder 120 as well as one or more (e.g., a pair, a ring, etc.) illumination sources 118. Ingestible device 400 may include and/or contain one or more power units 126, a transceiver 427, e.g. an RF transceiver, and one or more antennas 128a for transmitting data and one or more antennas 128b for receiving data, e.g. receiving control data. In other embodiments one or more antennas 128a and/or 128b may be used for transmission as well as reception. In other embodiments, transceiver 427 and or its functionality may be spread among a plurality of components. Transceiver 427 may include control and or processing capability, for example transceiver 427 may be or include a controller for controlling various operations of ingestible device 400, although control capability or one or more aspects of control may be included in a separate component such as for example circuit board or other circuitry included in ingestible device 400.

External recorder 412 may receive transmissions, such as, for example, a stream of wireless communication frames, from ingestible device 400 through one or more receiving antennas 15. Recorder 412 may include a signal selector 150 that may select or may produce an output signal that may be selected from the plurality of received signals and/or combined, a control unit 155 to control operation of recorder 412 and demodulate the received signals, a storage unit 157 to store data received, and a power unit 159 to power recorder 412.

According to an embodiment of the present invention, recorder 412 may include one or more transmitting antennas 16 to, for example transmit signals through the downlink channel to ingestible device 400. Transmitted signals may contain control data or other data that may be for exampled stored in storage unit 157 and/or processed in processing unit 155.

Interference detection for the uplink channel via antennas 15 may be similar to the discussion presented in reference to FIG. 1 describing the first embodiment of the present invention. In one embodiment of the present invention, control unit 155 may prompt recorder 412 during a period when the uplink channel is normally idle or during idle time between frames, to pick up signals that may be present in the surrounding environment. Signal selector 150, may for example select the strongest signal picked up from the plurality of antennas 15. Interference sensor 160 may determine the signal strength. The signal strength detected or any function or indication of signal strength level may be recorded in storage unit 157.

According to the fourth embodiment of the present invention, an interference sensor 560 for the downlink channel may be included and or contained within ingestible device 400, e.g. within the housing 110 of the ingestible device 400. For example, an antenna 128b may, e.g., during an idle period of the downlink channel, receive and/or pick up signals in the frequency range of antenna 128b from the surrounding environment that may be interfering with proper communication through the downlink channel. The strength of signals picked up, e.g. during the idle period of transmission, may be detected by an interference sensor 560. Interference sensor 560 may be for example a RSSI component to detect the strength of a signal received by antenna 128b. An amplifier 561 may be used to amplify the received signal prior to detecting the signal strength. In one example the amplifier 561 and/or the interference sensor 560 may be integral to the transceiver 427. In alternate examples, the interference sensor 560 may be separate or integral to other components, e.g. the imager 116 or other components in the ingestible device 200. Data detected by interference sensor 560 may be transmitted to receiver 412 during the regular transmission period of ingestible device 400. For example, signal strength data may be sent together with an image frame transmitted from ingestible device 400. For example, signal strength data may be included as a header of suffix of the transmitted image data. Other methods of incorporating signal strength data with data otherwise transmitted from ingestible device 400 may be used. In an alternate example, signal strength data may be transmitted separately. According one example, the signal strength measurement may be transmitted to recorder 412 and the data may be stored in storage unit 157. According to one example, specified threshold levels of interference may be detected by circuitry in recorder 212 and the threshold level may be stored. In another example, determination of threshold levels may be performed in the ingestible device 400 prior to transmission. For example, an indication of interfering signals may only be transmitted for signal interference above a defined threshold. Other suitable methods of detecting, storing and communication the level of interference may be implemented.

Indicator 170 may alert a patient or health profession that the patient is situated in an area where interference signals are being picked up the recorder 412. For example, indicator 170 may be a visual indicator, e.g. an LED, an audio indicator, e.g. a buzzer or other indicator e.g. a vibrator that may give indication to the patient that he is situated in an area where interference signals are being picked up the recorder 412. In reaction to the indication provided by indicator 170, the patient may move away from or possibly cease operation off an interfering RF transmitter.

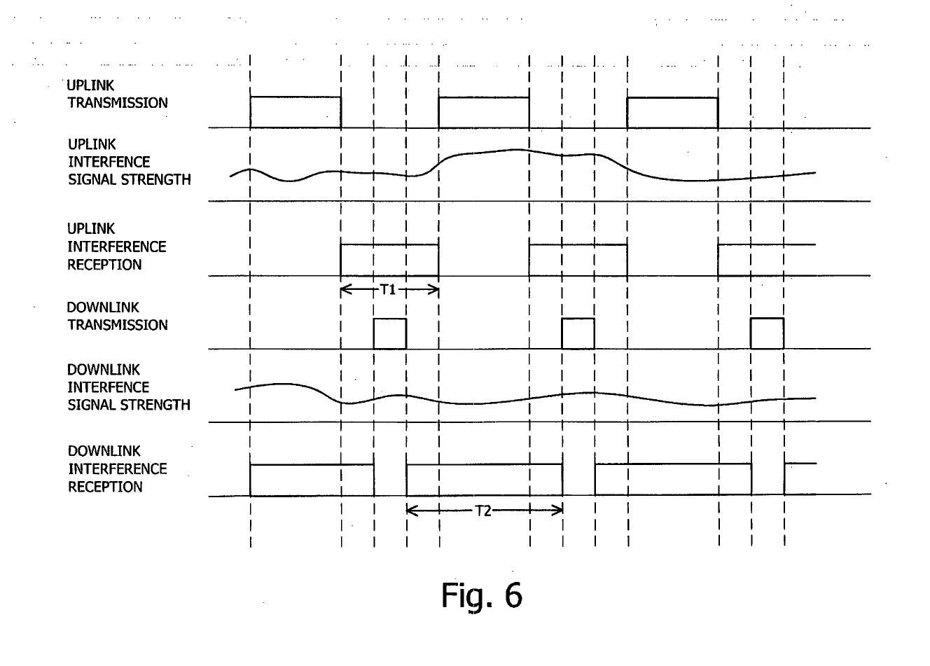

Reference is now made to FIG. 6 showing time diagrams of the uplink and downlink channels for ingestible device 300 and/or 400 and recorders 312 and/or 412 according to the third and fourth embodiment of the present invention. For simplification purposes only the time diagram for the third embodiment will be described herein. However, the same explanation may be applied to the fourth embodiment described herein. According to third embodiment of the present invention, the ingestible devices 300 may transmit data (uplink transmission) at a defined rate, for example every 500 msec. at a frequency range in the order of magnitude of 400 MHz. Interference signals in that frequency range, e.g. the uplink frequency range may be present in the vicinity of recorder 312. The recorders 312 may receive and record the data transmitted by ingestible device 300 together with interference signals. During a defined period, an idle period for transmission by the ingestible device, the interference signal may be picked up (reception of uplink interference signals) and there signal strength may be determined in the defined frequency range during a period T1. The start of the idle period for transmission may be prompted by the end of reception for a defined period before a subsequent reception is anticipated. In other embodiments interference signals may be detected in a selected period within period T1.

According to third embodiment of the present invention, the recoding device 300 may transmit data (downlink transmission) at a defined rate, for example every 500 msec. at a frequency range in the order of magnitude of 10 MHz. Interference signals in that frequency range, e.g. the downlink frequency range may be present in the vicinity of ingestible device 300 and/or recorder 312. Ingestible device 300 may receive and record the data transmitted by recorder 312 together with interference signals. During a defined period, an idle period for transmission by the recorder 312, the interference signal may be picked up (reception of downlink interference signals) and their signal strength may be determined in the defined frequency range during a period T2. The start of the idle period for transmission may be prompted by the end of transmission from the recorder 312 and for a defined period before a subsequent transmission is due. In other embodiments interference signals may be detected in a selected period within period T1. Due to the different frequency ranges used for the uplink and downlink channels, interference signals may be detected for example, in the downlink channel during transmission through the uplink channel and visa versa, interference signals may be detected for example, in the uplink channel during transmission through the downlink channel. Interference signals may be stored together with the data received from ingestible device 300 and/or may be processed before storing.

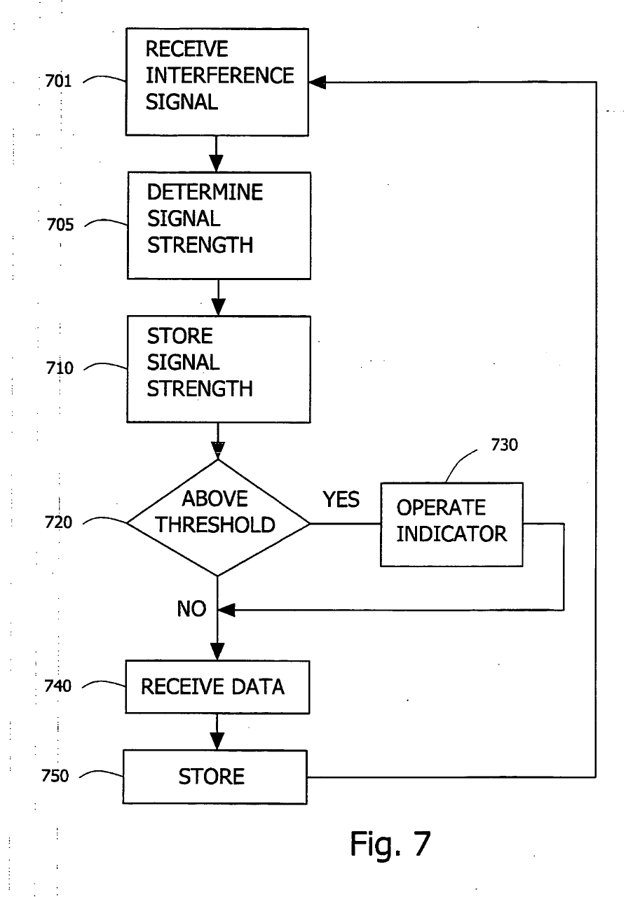

Reference is now made to FIG. 7 showing a flow chart describing a method for detecting interference levels in the uplink channel according to an embodiment of the present invention. The uplink channel may include transmission from the ingestible device 100, 200, 300, and/or 400 to the recorder 12, 212, 312, and/or 412. In block 701, the interference signal from the surrounding environment may be picked up by for example one or more of the recorder antennas 15 or by the antenna 128 and/or 128a of the ingestible device 100, 200, 300, and/or 400. According to one embodiment of the present invention the frequency range of the uplink interference signal picked up may be in the range of approximately 400 MHz. Other ranges may be used. Reception of interference signals may be performed, for example, in one or more time slots where the uplink channel transmission is idle. In other embodiments of the present invention, reception of interference signals may be prompted by the completion of the uplink transmission, for example after reception of a suffix line or after predetermined period of time elapsed from the start of the uplink transmission. Other methods of prompting reception of interference signals may be implemented. Received signals may be amplified and if more than one antenna was used to receive the interference signal, signal selection or combining may be performed as may be described herein. In block 705 the signal strength of the received interference, or alternately the signal strength of the selected interference signal or combined interference signal may be determined and or measured. For example an RSSI unit may be implemented for this purpose as may be described herein. Other methods may be used to determine signal strength levels. Signal strength detection may be performed in the ingestible device 100, 200, 300, and/or 400 or may be performed in the recorder 12, 212, 312, and/or 412. In block 710, the signal strength detected may be stored, for example, stored temporarily in the ingestible device 100, 200, 300, and/or 400 and/or stored in the recorder 12, 212, 312, and/or 412. In one example, signal strength measurements detected or any function or indication of signal strength level within the ingestible device 100, 200, 300, and/or 400 may be transmitted to the recorder 12, 212, 312, and/or 412, for example together with an uplink transmission signal and/or as may be described herein. In block 720 a threshold detector may determine, for example, if the interference level determined may be above a predetermined threshold. Thresholding may be performed either in the ingestible device 100, 200, 300, and/or 400 and/or in the recorder 12, 212, 312, and/or 412. For interference signals above a predetermined threshold, the indicator 170 may be operated (block 730) to for example indicate to the patient or the health professional that the systems is situated in an environment, region, and/or area with interference signals. The indicator 170 may be a visual, audio, or tactile indication as may be described herein. In one example, when thresholding is performed in the ingestible device 100, 200, 300, and/or 400, indication or the signal strength level may be transmitted to the recorder 12, 212, 312, and/or 412 only when the signal strength level may be above the threshold. In other examples, the signal strength level may always be transmitted or may be transmitted based on other considerations. In block 740 uplink channel transmission is received by the recorder 12, 212, 312, and/or 412 and in block 750 the uplink data transmitted may be stored. If the signal strength data was not previously stored in the receiver 12, 212, 312, and/or 412 (in block 710) the signal strength data may be stored together with the uplink transmitted data in block 750 and or may be stored during another time or time slot.

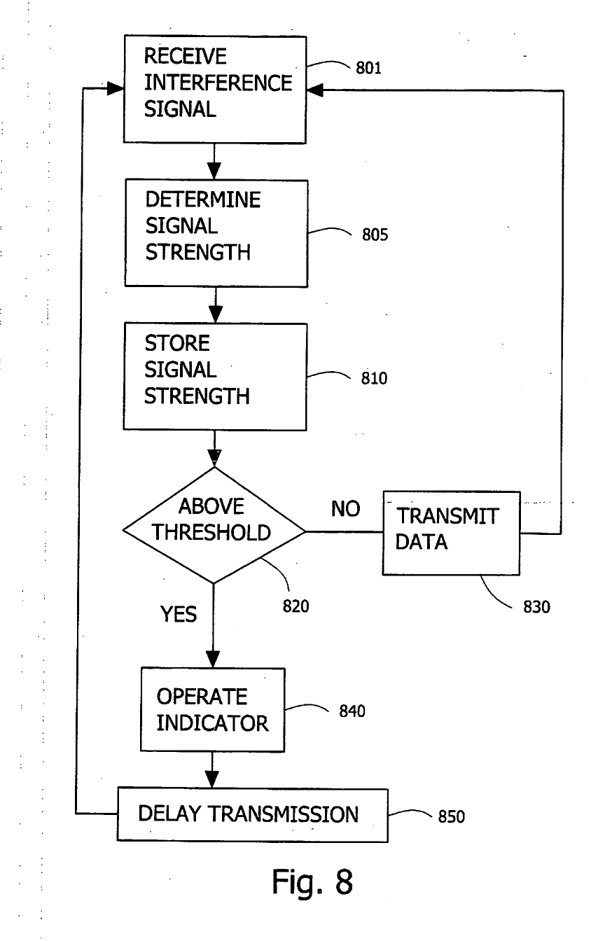

Reference is now made to FIG. 8 showing a flow chart describing a method for detecting interference levels in the downlink channel according to an embodiment of the present invention. The downlink channel may include transmission from the recorder 312 and/or 412 to ingestible device 300 and/or 400. In block 801, the interference signal from the surrounding environment may be picked up by for example one or more of the recorder antennas 16 or by the antenna 128b of the ingestible device 300 and/or 400. According to one embodiment of the present invention the frequency range of the downlink interference signal picked up may be in the range of approximately 10 MHz. Other ranges may be used. Reception of interference signals may be performed, for example, in one or more time slots where the downlink channel transmission is idle. In other embodiments of the present invention, reception of interference signals may be prompted by the completion of the downlink transmission. Other methods of prompting reception of interference signals may be implemented. Received signals may be amplified and if more than one antenna was used to receive the interference signal, signal selection or combining may be performed as may be described herein. In block 805 the signal strength of the received interference, or alternately the signal strength of the selected interference signal or combined interference signal may be determined and or measured. For example an RSSI unit may be implemented for this purpose as may be described herein. Other methods may be used to determine signal strength levels. Signal strength detection may be performed in the ingestible device 300 and/or 400 or may be performed in the recorder 312 and/or 412. In block 810, the signal strength detected may be stored, for example, stored temporarily in the ingestible device 300 and/or 400 and/or stored in the recorder 312 and/or 412. In one example, signal strength measurements detected within the ingestible device 300 and/or 400 may be transmitted to the recorder 312 and/or 412, for example together with an uplink transmission signal and/or as may be described herein. In block 820 thresholding may be performed to determine, for example, if the interference level determined may be above a predetermined threshold. Thresholding may be performed either in the ingestible device 300 and/or 400 and/or in the recorder 312 and/or 412. When interference levels may be below a predetermined threshold downlink channel transmission may be initiated and/or approved (block 830). For interference signals above a predetermined threshold, the indicator 170 may be operated (block 840) to for example indicate to the patient or the health professional that the systems is situated in an environment, region, and/or area with interference signals. The indicator 170 may be a visual, audio, or tactile indication as may be described herein. In one example, when thresholding is performed in the ingestible device 300 and/or 400, indication or the signal strength level may be transmitted to the recorder 312 and/or 412 only when the signal strength level may be above the threshold. In other examples, the signal strength level may always be transmitted or may be transmitted based on other considerations. In another example, the signal strength of transmission and/or reception may be increased when the signal strength of the interferer was determined to be above a defined threshold. Alternatively the signal strength of transmission and/or reception may be decreased when the signal strength of the interferer was determined to be below a defined threshold. In block 850, transmission may be delayed in situations when the signal strength of the interfering signal may be above a defined threshold. Transmission delay may by methods known in the art and/or by methods described herein. If the signal strength data was not previously stored in the receiver 312 and/or 412 (in block 810) the signal strength data may be stored together with the uplink transmitted data or may be stored during another time or time slot. Reception of interference signals (block 801) may continue until proper reception can be insured.

In another embodiment of the present invention, interference level measurement may be performed during time stamp recording. Time stamp recording may be performed periodically in for example, the recorder whenever no information may be received from the ingestible device 100. Time stamp recording may include recording battery consumption, temperature, interference level and other parameters of the recorder 12. Time stamp recording may aid in determining malfunction of either the ingestible device 100 and/or recorder 12. Interference level may be an important parameter for determining the working condition of the ingestible device 100 and/or recorder 12. For example, recorder 12 may fail to receive transmissions from ingestible device 100 due to high interference levels. As a result it may record periodical time stamps which include among other parameters also the signal strength level. In case high signal strength of the interfering signal is measured it can be a result of strong interference. In such a case the recorder may give indicate with indicator 170 by special LED colors, buzzer, vibration or other means of indication.

While the present invention has been described with reference to one or more specific embodiments, the description is intended to be illustrative as a whole and is not to be construed as limiting the invention to the embodiments shown. It is appreciated that various modifications may occur to those skilled in the art that, while not specifically shown herein, are nevertheless within the true spirit and scope of the invention.

1. A system for sensing interference in a wireless communication channel between an ingestible imaging device and an external recorder comprising:
an ingestible imaging device wherein the ingestible imaging device transmits a signal by wireless communication;
a recorder, wherein the recorder receives a signal by wireless communication; and
an interference sensor, wherein the interference sensor senses a level of interference in the wireless communication channel.

2. The system according to claim 1 wherein the interference sensor comprises a signal strength detector to detect the strength of an interfering signal.

3. The system according to claim 2 wherein the signal strength detector comprises an RSSI unit.

4. The system according to claim 1 wherein the interference sensor includes a threshold detector to detect a level of interference above a defined threshold.

5. The system according to claim 1 comprising an indicator wherein the indicator is to give a user indication of a level of interference in a communication channel.

6. The system according to claim 5 wherein the indicator is a visual indicator.

7. The system according to claim 1 wherein the wireless communication is in a radio frequency range.

8. The system according to claim 1 wherein the wireless communication is in a frequency range between 10-15 MHz.

9. The system according to claim 1 wherein the wireless communication is in a frequency range between 400-450 MHz.

10. The system according to claim 1 wherein the interference sensor is contained within the ingestible imaging device.

11. The system according to claim 1 wherein the interference sensor is contained within the recorder.

12. The system according to claim 1 wherein the ingestible imaging device comprises a transceiver to transmit and receive a signal by wireless communication.

13. The system according to claim 12 wherein the transceiver transmits a signal at a first frequency range and receives a signal at a second frequency range and wherein the interference sensor senses interference in at least one frequency range.

14. The system according to claim 1 wherein the interference sensor is to be operated during an idle time of signal transmission by the ingestible imaging device.

15. The system according to claim 1 wherein the interference sensor is to be operated during an idle time of signal reception by the ingestible imaging device.

16. A method for sensing interfering signals in a wireless communication channel between an ingestible imaging device and an external recorder, the method comprising:
sensing an interference signal during an idle time of communication in a communication channel;
determining the strength of the interference signal; and
recording the level of interference on an external recorder.

17. The method according to claim 16 comprising determining the threshold level of the strength of the interference signal.

18. The method according to claim 16 comprising operating an indicator upon detection of an interference level that is above a defined threshold.

19. The method according to claim 16 comprising delaying transmission of a signal in a communication channel upon detection of an interference signal above a defined threshold.

20. The method according to claim 16 comprising sensing the interference signal during time stamping of the external recorder.

21. The method according to claim 16 comprising transmitting interference data from the ingestible device to the external recorder.

22. The method according to claim 16 comprising storing interference data on the external recorder.

23. The method according to claim 16 comprising altering the strength of transmission in a communication channel based on the determined strength of the interference signal.
